(19) United States
(12) Patent Application Publication
(10) Pub. No.: US 2024/0081723 A1
(43) Pub. Date: Mar. 14, 2024

(54) ALLERGY TEST DEVICE

(71) Applicant: ALLTEST GMBH, Linz (AT)

(72) Inventor: Guntram SCHNETZ, Biedermannsdorf (AT)

(73) Assignee: ALLTEST GMBH, Linz (AT)

(21) Appl. No.: 18/272,617

(22) PCT Filed: Jan. 12, 2022
(86) PCT No.: PCT/AT2022/060005

§ 371 Date: Jul. 17, 2023

(30) Foreign Application Priority Data
Jan. 15, 2021 (AT) A 50010/2021

(51) Int. Cl. A61B 5/00; A61B 10/00; A61M 37/00

(52) U.S. Cl. CPC A61B 5/411; A61B 10/0035; A61M 37/0015

(57) ABSTRACT

An allergy test device for applying a liquid medium onto or into the skin, having a film with a flat part for placing on the skin and with at least one part which is spaced apart from the skin during use and has a container for receiving the liquid medium, wherein each container of each spaced-apart part has at least one closable opening. Each spaced-apart part is designed to be deformable in the direction of the flat part of the film, and a valve is arranged movably in the at least one opening of each container, wherein the opening of the container is sealed off in a closure position of the valve, and, upon deformation of the spaced-apart part, the valve is at least partially opened in a position deviating from the closure position.

### CROSS-REFERENCE TO RELATED APPLICATIONS

The present application is a national phase application of PCT Application No. PCT/AT2022/060005, filed Jan. 12, 2022, entitled “ALLERGY TEST DEVICE”, which claims the benefit of Austrian Patent Application No. A 50010/2021, filed Jan. 15, 2021, each of which is incorporated by reference in its entirety.

### BACKGROUND OF THE INVENTION

### 1. Field of the Invention

The invention relates to an allergy test device for applying a liquid medium to or into the skin, with a film with a flat part for laying on the skin and at least one part spaced apart from the skin during use with a container for holding the liquid medium, wherein each container of each spaced part has at least one closable opening.

### 2. Description of the Related Art

In the past, the prick test involved a very elaborate and time-consuming way of dripping various allergens manually onto the skin, for example on the forearm or back, and puncturing the skin so that the substance in question could penetrate the epidermis. After a specified testing time, the allergic reaction was assessed by determining the reddening of the skin and the size of the weal. A positive control with histamine and a drug-free negative control are usually carried out in parallel. In addition to the great expenditure of time for the medical staff and the patient or subject, it is very difficult, particularly in the case of very young patients or subjects, to endure the duration of the treatment without moving. In addition, there is a risk, particularly when the patient or subject moves, that the liquid media that have been dripped on will be smudged or mixed.

With the help of allergy test devices which already contain the liquid media to be investigated, the performance of such allergy tests or skin prick tests has been made considerably easier for both the medical staff and the patient or subject.

For example, US 2014/276196 A1 describes an allergy test device in the form of a film with a number of containers for holding various liquid media, which is stuck onto the skin. After it has been applied, a rocker at the top of the film applies pressure over all containers, piercing the underside of the film while simultaneously puncturing the top layer of skin and applying the liquid medium to the puncture site.

Other constructions of allergy test devices are known, for example, from U.S. Pat. No. 2,841,138 A or EP 2 163 203 A1.

An allergy test device in the form of a blister strip is described in AT 515 791 B1, wherein two films are provided and the respective liquid medium is contained in protuberances in the upper film. By manual pressure on the protuberances, the medium contained therein is pressed through an opening onto the surface of the skin and, if necessary, the skin is simultaneously scratched or punctured with pointed or sharp elements, so that the medium can penetrate the skin and possibly cause an allergic reaction there.

Known devices for carrying out allergy tests usually do not permit a defined application of the respective medium to the skin or into the skin, since the opening of the container holding the medium depends heavily on the direction and magnitude of the compressive force exerted. This does not result in good reproducibility of the allergy tests.

In addition, many allergy test devices have a very complicated structure and are laborious to manufacture or to fill with the liquid media, which increases costs.

### SUMMARY OF THE INVENTION

The object of the present invention is therefore to create an allergy test device as mentioned above which can be produced as simply and inexpensively as possible and consists of as few components as possible. In addition, flawless functioning and reproducibility of the allergy tests should be guaranteed and the liquid medium contained in the containers should be able to be applied to the surface of the underlying skin in a safe and defined manner, so that reliable statements can be made about any allergic reactions. Disadvantages of known allergy test devices should be avoided or at least reduced.

The present invention relates to allergy test devices with at least one spaced part with at least one container for holding a liquid medium, but in particular with several spaced parts with several containers for holding different liquid media. As a result, allergy tests for several allergens can be carried out simultaneously in a particularly efficient manner that saves time for the patient or subject.

The present invention relates to allergy testing devices used for testing an allergic reaction by merely applying the respective liquid medium to the surface of the skin without injuring the skin. For example, an allergic reaction to certain cosmetics, such as ointments or creams, can be determined in this way. Also included are allergy test devices for carrying out a so-called prick test, in which the skin is punctured or scratched, so that the liquid medium to be tested can penetrate the skin and possibly cause an allergic reaction.

The term liquid media includes a wide variety of liquids which contain ingredients which are to be examined with regard to an allergic reaction on the part of the patient or subject. For example, such ingredients may be defined allergen extracts, such as pollen extracts, food extracts, etc. Low-viscosity media are particularly suitable for use, but the use of viscous or gel-type media is also conceivable, provided they can be applied to or into the skin. Components in solid form, for example freeze-dried allergens in the form of a lyophilisate, which are mixed with the liquid medium when the allergy test device is used, can also be arranged at suitable points on the allergy test device. The mixture of two or more components is then applied to or into the skin.

The object of the invention is achieved in that each spaced part is designed to be deformable in the direction of the flat part of the film and that a valve is slidably arranged in the at least one opening of each container, wherein the opening of the container is sealed in a closed position of the valve and on deformation of the spaced part, the valve is at least partly opened in a position deviating from the closed position. The allergy test device according to the invention consists of few components which are relatively easy to produce, for example by injection molding from suitable plastics. Assuming the production of correspondingly large quantities, relatively low production costs can also be achieved. By arrangement of a valve in the opening of each container with the liquid medium, a defined opening of at least part of the opening of the container can take place when pressure is applied to the spaced part of the film in the direction of the skin surface, and thus a safe application of the liquid medium and any substances mixed with it to the skin surface. By appropriate design of the valve and the opening of the container, the container can be sealed when the valve is in the closed position, and the liquid medium in the container can thus be safely stored, even over long periods of time. Depending on the medium used, the container can also be filled with it just before the allergy test is carried out. Ideally, however, the allergy test devices are already pre-filled with the liquid medium and any solid substances separated from it, such as freeze-dried allergens (lyophilisates), which are mixed with the liquid medium during use, and sold in appropriate packaging so that the medical staff only has to open them and use them on the patient or subject.

The container is preferably integral with the spaced part of the film. As a result, the production costs can be minimized even further, since both the film and the container can be produced from one material and preferably also from one piece.

In the case of a container integral with the spaced part of the film, a filling opening, preferably closable with a lid, can be arranged in each spaced part for filling the container with the liquid medium. Although this makes the manufacturing process more complex and thus the manufacturing costs higher, it makes it much easier to fill the containers of the allergy test device with the respective liquid medium. Instead of being introduced via a filling opening, the medium could also be introduced into the container with the aid of a syringe through a puncture membrane, for example.

As an alternative to the above-described integration of the container with the spaced part of the film, the container can also be manufactured separately, essentially in the form of a cup, and subsequently connected appropriately to the spaced part of the film. This variant facilitates the manufacture and formation of the film, but requires an additional manufacturing step of connecting the container to the spaced part of the film. However, the process of filling the container with the respective liquid medium can be ideally integrated into this additional manufacturing step.

Preferably, at least each spaced part of the film, preferably the entire film, is designed to be elastically deformable. This ensures that the spaced part is moved back to its original position after the pressure has been applied, thereby moving the components of the allergy test devices away from the skin surface again after the liquid medium or a mixture of the liquid medium with solid substances from the container has been applied to the skin or introduced into the skin. This means that any allergic reaction to the medium in question can take place unimpeded while the allergy test is being carried out and an optimal result can be achieved.

The film is preferably made of synthetic material, in particular thermoplastic synthetic material or silicone. These materials are easy to process and can also meet biocompatibility requirements. In addition, elastic deformability, i.e. return to the original shape or at least essentially the original shape after deformation, can be achieved with such materials.

Each spaced part can be formed by a protuberance of the film, so that a preferably closed cavity to the skin is formed during use. As a result, mixing of the liquid media from different protuberances during the allergy test and an influence of external influences on the examination can be prevented. Provided that the design is suitable, movement of the patient or subject person during the allergy test can even be made possible within certain limits, since the liquid medium cannot run out in an uncontrolled manner.

In order not to prevent the container from emptying into the cavity below the protuberance of the film, a vent hole can also be arranged in the protuberance. When the protuberance is compressed, air escapes via the vent hole, which can contain a valve, if need be, so that no air from the outside can get into the cavity, as a result of which the liquid medium can flow unhindered from the container onto the skin surface.

To carry out a so-called prick test, at least one pointed or sharp element for local and superficial injury to the skin is provided on the side of the container and/or the valve that faces the skin during use. The at least one pointed or sharp element is arranged at a suitable point or points on the underside of the container and/or the valve, whereby during the deformation of the spaced part of the film, the appropriate application of pressure at the same time as the opening of the valve also punctures or scores the skin.

Advantageously, the at least one pointed or sharp element is made in one piece with the container and/or the valve. For example, the pointed or sharp element can be integrated into the injection molding process when the container or valve is produced, whereby the production costs can be kept very low. For special applications it may also be necessary to use pointed or sharp elements made of stainless steel, which can also be arranged later on the container or valve, but this increases the manufacturing costs.

According to a further feature of the invention, each valve is formed by an essentially cylindrical body with at least one channel, which cylindrical body seals the opening of the container in the closed position of the valve and opens the at least one channel to the container in a position deviating from the closed position. Such a cylindrical body is relatively easy to manufacture.

The at least one channel can be formed by at least one channel running inside the cylindrical body with a connection to at least one opening at the lateral surface. In the closed position, the opening of the channel running inside is closed at the lateral surface and no liquid medium can escape from the container to the outside. When the valve is moved out of the closed position, the at least one opening at the lateral surface of the cylindrical body is opened, whereby the liquid medium can reach the surface of the skin through the channel running in the interior by means of this opening. In addition, a solid substance can be arranged inside the cylindrical body or the interior of the cylindrical body can be provided with a coating of a solid substance, for example a freeze-dried allergen (lyophilisate), which solid substance is mixed with the liquid medium during use of the allergy test device and applied to the skin or introduced into the skin.

Alternatively or additionally, the at least one channel can also be formed by at least one channel running on the surface of the cylindrical body. In the closed position of the valve, the opening of the container is sealed by appropriate constructions of the valve and the opening and the liquid medium is prevented from escaping from the container. When the valve is moved out of the closed position, the at least one channel on the surface of the cylindrical body is opened and the liquid medium flows from the container via the at least one channel running outside at the surface of the valve towards the skin.

A sealing lip for sealing the opening of the container in the closed position can be integrated with the cylindrical body of the valve. Such a sealing lip can easily be integrated into the manufacturing process of the valve, for example the injection molding process. The sealing lip reliably prevents unintentional escape of the liquid medium from the container and unintentional mixing of the liquid medium with any solid substance present when the valve is in the closed position.

An adhesive film with a peel-off film is preferably arranged on the side of the flat part of the film that faces the skin during use. Thus, the appropriately prefabricated allergy test device can be used quickly by simply peeling off the peel-off film and sticking it to a suitable area of the patient's or subject's skin. Instead of a prefabricated adhesive film, the allergy test device can also be prepared for use on the patient or subject by applying, in particular spraying, a suitable adhesive to the underside of the flat parts of the film. In addition, the allergy test device can also be easily attached and fixed to the skin surface with adhesive strips.

According to a further feature of the invention, several spaced parts of a film are arranged in at least two rows. Thus, an allergic reaction can occur simultaneously to different substances contained in the liquid media of the different containers in the spaced parts of the film.

In order to permit unambiguous assignment of the respective liquid media in the containers of the individual spaced parts, the number of spaced parts of a film is different in each row. This makes it possible to prevent the rows from being mixed up when the allergic reactions are assessed, and to achieve reliable and unambiguous assignment of the respective liquid media to the corresponding skin areas.

An identification is preferably placed on the outside of each spaced part. For example, numbers or letters are arranged on the outside of the spaced parts or protuberances of the film, wherein the liquid media are assigned to these numbers or letters in an accompanying legend. As a result, the medical personnel know which liquid medium is contained in which container of which spaced part of the film and, after the allergy test has been carried out, can quickly and easily assign an allergic reaction to the respective liquid medium or the substance dissolved therein.

The film can be formed at least partly from transparent material, for example from transparent thermoplastic synthetic material or transparent silicone. By this means, on the one hand, the filling of the containers and, on the other hand, the correct implementation of the allergy test can be inspected and checked. If not all parts are made of transparent material, the latter can be produced, for example, in a two-component injection molding process after the production of the non-transparent parts.

If a transport lock is provided, with essentially cylindrical elements which are designed to be arranged on the underside of the film for enclosing the containers and valves in the spaced parts, unintentional displacement of the valves when the spaced parts of the film are deformed and thus unintentional opening of the containers and leakage of the liquid media from the containers and mixing of the liquid medium with any solid substances present can be prevented. The transport lock can also be produced very easily and cheaply, preferably using the injection molding process. The allergy test device is preferably delivered together with such a transport lock and preferably in packaging, and the transport lock is only removed shortly before the allergy test device is used and shortly before the device is stuck to the skin surface of the patient or subject.

If a ruler, preferably with semi-circular recesses with measuring lines, is arranged on the transport lock for measuring allergic reactions on the skin, the evaluation of the allergy test can be simplified or supported. Medical personnel can use the ruler after removing the allergy test device from the patient's or subject's skin surface to determine the size of the weals produced by the allergic reaction. For this purpose, several semi-circular recesses, for example, are provided on the ruler with different diameters, which are brought into correspondence with the allergic reaction in order to be able to read off the size of the circular reddening. Different shades of red for quantifying skin redness could also be printed on the ruler of the transport lock to facilitate the medical staff's diagnosis.

If a preferably sterile packaging is provided, the allergy test device can be kept clean or sterile until it is filled with the liquid media and any solid substances or, in the case of already filled containers, also until it is used on the patient or subject. To make it possible to view the allergy test device in the packaging, the packaging is at least partly transparent. Any sterilization of the allergy test device can be carried out using suitable sterilization methods, such as gas, gamma radiation, UV light, etc.

The film, the container, the valve and/or the at least one pointed or sharp element are preferably made at least partially from biocompatible material, for example polyether ether ketone (PEEK), or are at least partially coated with a biocompatible coating, for example a parylene coating.

### BRIEF DESCRIPTION OF THE DRAWINGS

The invention is explained in more detail with reference to the attached drawings. These show:

FIG. 1A—a schematic representation of the principle of the allergy test device according to the invention in a sectional side view in the closed position of the valve, in which the opening of the container is sealed;

FIG. 1B—the allergy test device according to FIG. 1A in a position that deviates from the closed position of the valve, in which the opening of the container for releasing the liquid medium is at least partly opened;

FIG. 2—a sectional view through an embodiment of the allergy test device according to the invention with a container integrated with the film;

FIG. 3A—a sectional view through a further embodiment of the allergy test device according to the invention in the closed position of the valve;

FIG. 3B—the allergy test device according to FIG. 3A in a position for releasing the liquid medium that deviates from the closed position of the valve;

FIG. 4A—a sectional view through a further embodiment of the allergy test device according to the invention in the closed position of the valve;

FIG. 4B—the allergy test device according to FIG. 4A in a position for releasing the liquid medium that deviates from the closed position of the valve;

FIG. 5—a perspective view of a transport lock with an integrated ruler for measuring the allergic reaction;

FIG. 6—a sectional view through a variant of the allergy test device with a transport safety device arranged thereon;

FIG. 7—a detail VII from FIG. 6 in magnification;

FIG. 8—a perspective plan view of an embodiment of an allergy test device according to the invention from above; and

FIG. 9—a perspective view of an allergy test device arranged in packaging.

### DETAILED DESCRIPTION

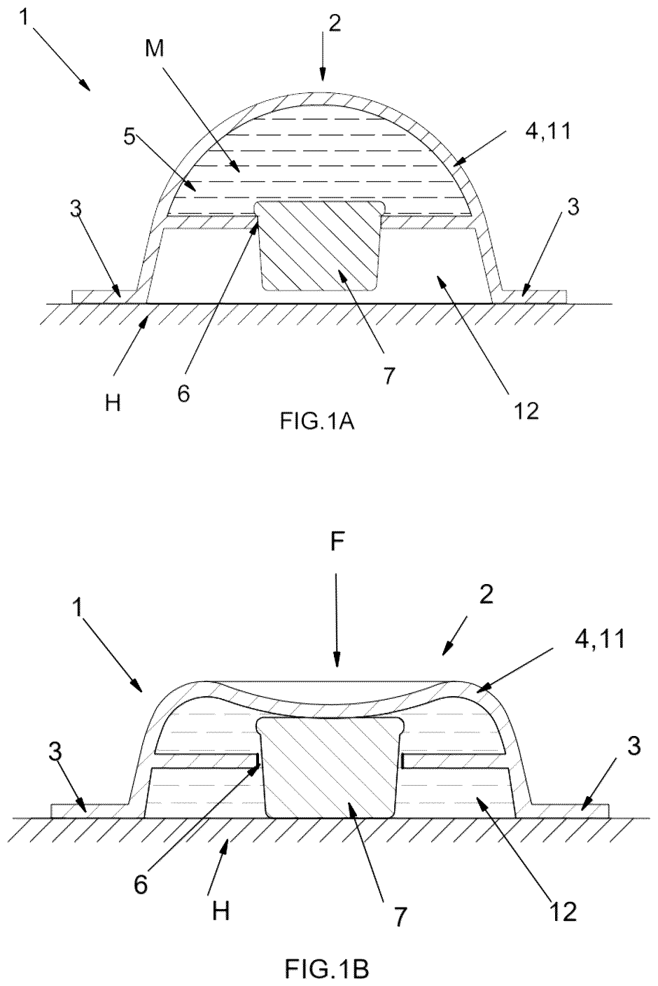

FIG. 1A shows a schematic representation of the principle of the allergy test device 1 according to the invention for applying a liquid medium M onto or into the skin H of a patient or subject. The allergy test device 1 has a film 2 with a flat part 3 for resting on the skin H and a part 4 spaced apart from the skin H during use. The film 2 can be formed at least partly from transparent material, for example from transparent thermoplastic synthetic material or transparent silicone. Each spaced part 4 of the film 2 has a container 5 for receiving the liquid medium M, the allergic reaction to which is to be tested. The medium M can also be viscous or in the form of a paste or gel. Each container 5 in the spaced part 4 has at least one closable opening 6 through which the liquid medium M can be applied to the skin H or introduced into the skin H. The flat part 3 of the film 2 can be fixed on the surface of the skin H with an adhesive or an adhesive film (not shown). According to the invention, each spaced part 4 is designed to be deformable in the direction of the flat part 3 of the film 2. A valve 7 is slidably arranged in the at least one opening 6 of each container 5. In FIG. 1A, the valve 7 is in its closed position, in which the opening 6 of the container 5 is sealed and the liquid medium M is prevented from escaping from the container 5.

If, according to FIG. 1B, a deformation of the spaced part 4 and of the container 5 is caused by the application of a force F to the spaced part 4 of the film 2, the valve 7 is brought into a position deviating from the closed position according to FIG. 1A as soon as it is present on the surface of the skin H and is thereby displaced into the interior of the container 5. The displacement of the valve 7 opens the opening 6 of the container 5 at least partly, whereby the liquid medium can escape from the container 5 onto the surface of the skin H. In the illustrated schematic example, the valve 7 is formed by a frustum-shaped plug, which opens an annular gap between the valve 7 and the opening 6 when it is moved out of the closed position, through which the liquid medium M can escape from the container 5. When the force F is removed, the spaced part 4 of the film 2 preferably returns to the original shape shown in FIG. 1A. For this purpose, at least the spaced part 4 of the film 2, preferably the entire film 2, is made of elastically deformable material. This prevents components of the allergy test device 1, such as the valve 7, from resting on the surface of the skin H and preventing or influencing the allergic reaction to the liquid medium M.

Such an allergy test device 1 can be manufactured relatively easily, preferably from suitable plastics by injection molding. The arrangement of the valve 7 in the opening 6 of the container 5 with the liquid medium M can result in a defined opening of at least part of the opening 6 of the container 5 when pressure is applied to the spaced part 4 of the film 2 in the direction of the surface of the skin H, and thus a safe application of the liquid medium M to the skin surface can be achieved. In the illustrated variant according to FIGS. 1A and 1B, the container 5 is integrated with the film 2 or the spaced part 4 of the film 2 and is manufactured with it in one piece. Here, the spaced part 4 is formed by a protuberance 11 of the film 2, so that when the allergy test device 1 is used, a preferably closed cavity 12 to the skin H is formed. However, the spaced part 4 of the film 2 does not necessarily have to be closed.

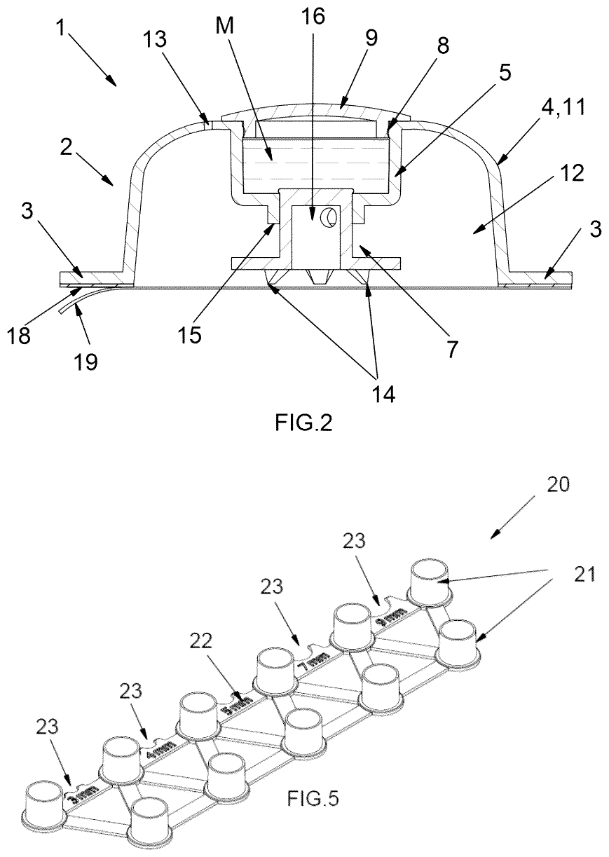

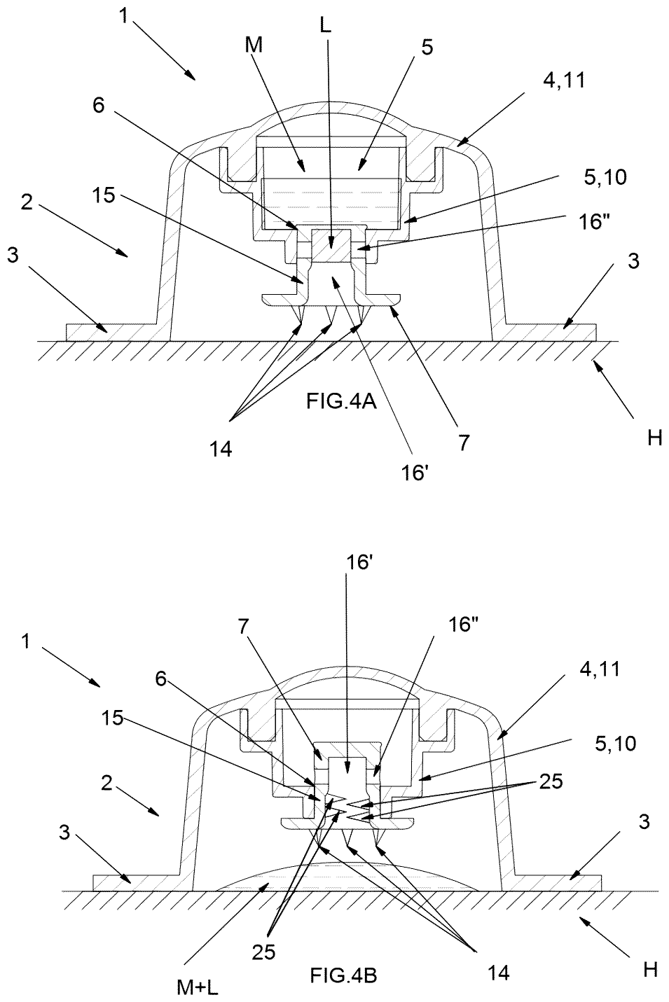

FIG. 2 shows a sectional view through another embodiment of the allergy test device 1 according to the invention with a container 5 integrated with the film 2. The spaced part 4 of the film is also formed here as a protuberance 11, whereby a closed cavity 12 to the skin surface forms. The container 5 is formed as a cup from the spaced part 4 of the film 2 and has a filling opening 8 which can be closed with a lid 9 for filling the container 5 with the liquid medium M. In the spaced part 4 of the film 2 or in the protuberance 11, a vent hole 13, possibly with a ventilation valve (not shown), can also be provided. The valve 7 is formed by an essentially cylindrical body 15 having a channel 16 running in it (see FIGS. 4A and 4B). On the side of the valve 7 that faces the skin H during use, pointed or sharp elements 14 are provided for local and superficial injury to the skin H, which are preferably produced in one piece with the valve 7. An adhesive film 18 with a peel-off film 19 can be arranged on the side of the flat part 3 of the film 2 that faces the skin H during use, as indicated schematically.

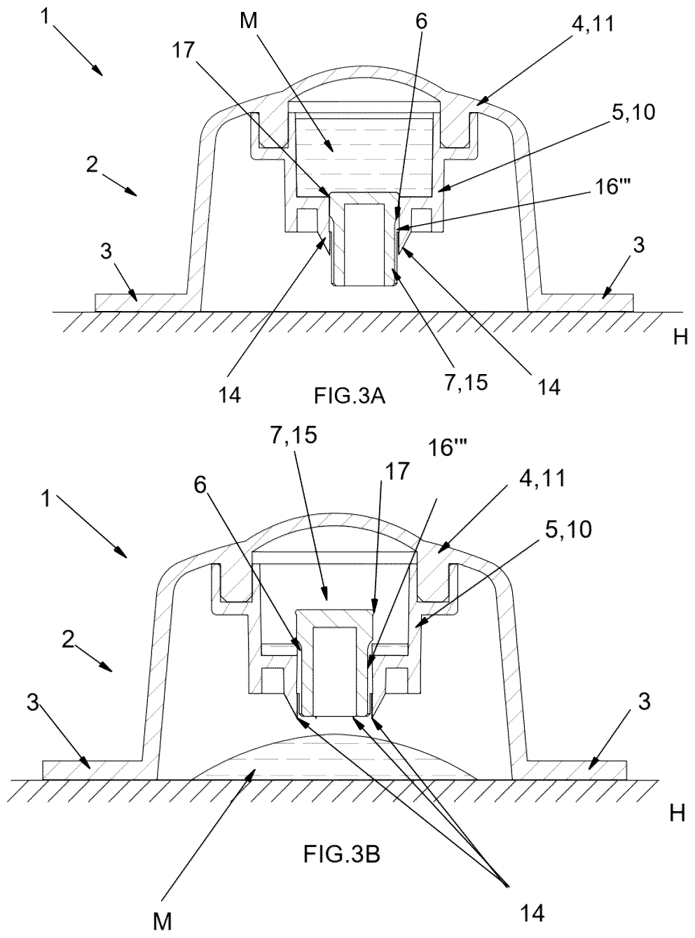

FIG. 3A shows a sectional view through a further embodiment of the allergy test device 1 according to the invention with the valve 7 in the closed position. Here the container 5 is formed by an independent body essentially in the form of a cup 10 and is connected appropriately tightly to the spaced part 4 of the film or the protuberance 11, for example by gluing. In the opening 6 of the container 5, a valve 7 is arranged, which is formed by an essentially cylindrical body 15 with channels 16′″ on the surface of the cylindrical body 15. These channels on the surface of the cylindrical body 15 forming the valve 7 can be formed by one or several axial grooves. In the closed position of the valve 7, the opening 6 of the container 5 is sealed by an extension of the diameter of the cylindrical body 15. In addition or as an alternative to this, a sealing lip 17 can also be arranged at the upper end of the cylindrical body 15 facing away from the skin H, which sealing lip can be easily produced in particular in an injection molding process.

If, according to FIG. 3B, the valve 7 is moved out of the closed position according to FIG. 3A by deformation of the spaced part 4 of the film 2, the channels 16′″ on the outside of the cylindrical body 15 of the valve 7 are opened and the liquid medium can run from the container 5 onto the surface of the skin H. Pointed or sharp elements 14, which serve to puncture or score the skin H, can be arranged on the side of the container 5 facing the skin H. The pointed or sharp elements 14 are preferably integral with the container 5 and manufactured in one piece with it.

FIG. 4A shows a sectional view through a further embodiment of the allergy test device 1 according to the invention with the valve 7 in the closed position. In contrast to the embodiment according to FIGS. 3A and 3B, the valve 7 is formed by an essentially cylindrical body 15 with a channel 16′ inside the cylindrical body 15 with a connection to at least one opening 16″ on the lateral surface of the cylindrical body 15. Pointed or sharp elements 14 are arranged on the side of the valve 7 facing the skin H and are preferably made in one piece with the valve 7. In addition, in this exemplary embodiment, a lyophilisate L (for example, a freeze-dried allergen) is arranged in the channel 16′ inside the cylindrical body 15, which is mixed with the liquid medium M and thereby activated when the valve 7 is moved out of the closed position (see FIG. 4B). The lyophilisate L can be preformed, pressed, chemically/physically bound or encapsulated. Instead of a preformed plug of the lyophilisate L, coating the inner wall of the channel 16′ or other suitable points of the allergy test device 1 with the lyophilisate L is also conceivable (not shown).

If according to FIG. 4B the valve 7 is moved out of the closed position according to FIG. 4A by deformation of the spaced part 4 of the film 2, the openings 16″ on the lateral surface of the cylindrical body 15 of the valve 7 are opened and the liquid medium can run from the container 5 through the openings 16″ to enter the channel 16′ running inside the cylindrical body 15, where the liquid medium M is mixed with the lyophilisate L. To support the mixing of the liquid medium M with the lyophilisate L, moldings 25 can be arranged on the inner wall of the cylindrical body 15. The mixture of the liquid medium M with the lyophilisate L then reaches the skin H or penetrates the skin H. The pointed or sharp elements 14 for puncturing or scoring the skin H are arranged on the side of the valve 7 facing the skin H.

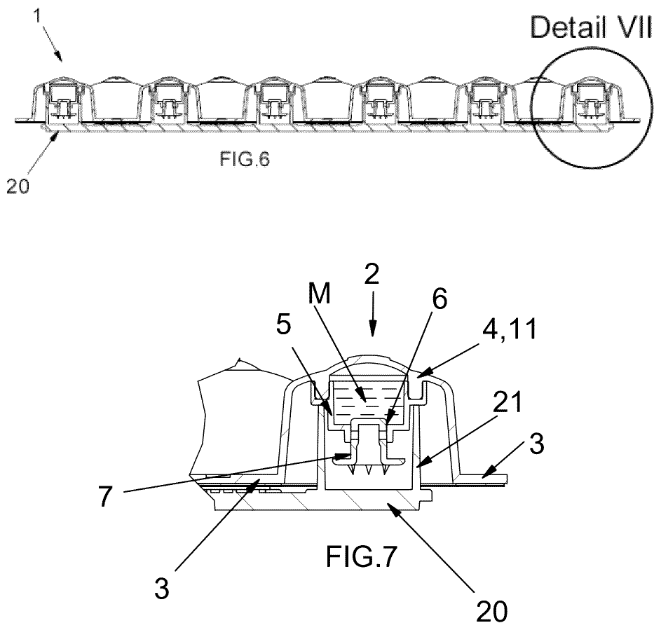

FIG. 5 shows a perspective view of a transport lock 20 for the allergy test device 1. The transport lock 20 has essentially cylindrical elements 21 placed according to the number and arrangement of the spaced parts 4 or protuberances 11 of the allergy test device 1, which are designed to be arranged on the underside of the film 2 for enclosing the containers 5 and valves 7 in the spaced parts 4 of the allergy test device 1 (see FIGS. 6 and 7). A ruler 22, preferably with semi-circular recesses 23 of different diameters, can be arranged on the transport lock 20 for measuring allergic reactions on the skin H. The respective diameters of the semi-circular recesses 23 can be printed to facilitate determination of the size of the weals of the allergic reaction.

FIG. 6 shows a sectional view through a variant of the allergy test device 1 with the transport lock 20 according to FIG. 5 arranged thereon.

FIG. 7 shows detail VII from FIG. 6 in magnification. The essentially cylindrical elements 21 protrude into the protuberances 11 of the film 2 and surround the underside of the container 5 located therein with the valves 7 slidably arranged in the opening 6. Because the spaced part 4 of the film rests on the free end of the cylindrical element 21, deformation of the spaced part 4 and hence unintentional displacement of the valve 7 from the closed position is prevented. Thus, unintentional outflow of the liquid medium M from the container can be effectively prevented. Before the allergy test is carried out, the transport lock 20 is detached from the allergy test device 1, whereby deformation of the spaced parts 4 of the film 2 for releasing the liquid media M onto the skin H is made possible. The transport lock 20 can simply be plugged onto the allergy test device 1 or it can also be attached to the flat part 3 of the film 2 with adhesive bonds.

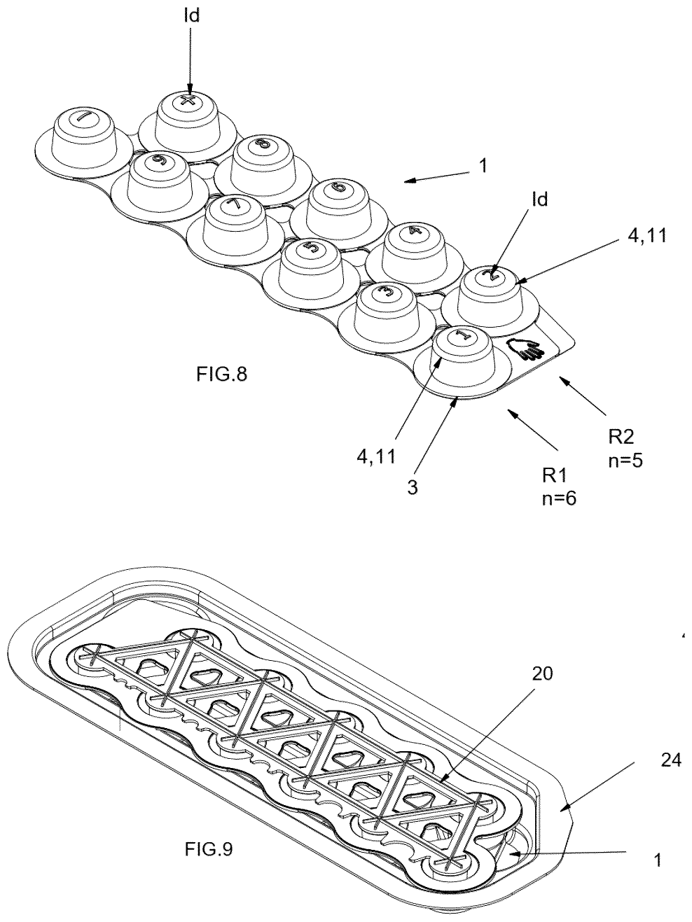

FIG. 8 shows a perspective plan view of an embodiment of an allergy test device 1 according to the invention from above. In this case, several spaced parts 4 of the film 2 or protuberances 11 are arranged in two rows R₁ and R₂. The number n₁ and n₂ of the protuberances 11 in each row R₁ and R₂ is deliberately designed to be different in order to permit easier and unambiguous assignment of the respective liquid media M and to facilitate the diagnosis of the allergy test. In the exemplary embodiment shown, six (n₁=6) spaced parts 4 with six containers 5 for six different liquid media M, i.e. six different allergy tests, are arranged in the first row R₁, while five (n₂=5) spaced parts 4 with five containers 5 for five liquid media M, i.e. five different allergy tests, are arranged in the second row R₂. Overall, the allergic reaction to eleven different substances can be tested simultaneously with this allergy test device 1, or at least to nine different substances if two containers 5 are reserved for a positive control with histamine and a negative control free of active ingredient.

An identification Id, for example a number or a letter, can be arranged on the upper side of the protuberances 11 for clear assignment, or a labeling field can be placed.

FIG. 9 shows a perspective view of an allergy test device 1 arranged in packaging 24. In the example shown, the allergy test device 1 with the transport lock 20 arranged on it can be seen from below. The packaging 24 is preferably at least partly transparent to make it possible to view the allergy test device 1. The allergy test device 1 can be sterilized before it is placed in the packaging 24 or afterwards using common sterilization methods.

The individual components of the allergy test device 1, such as the film 2, the containers 5, the valves 7 and/or the pointed or sharp elements 14 are preferably made of biocompatible material, for example polyether ether ketone (PEEK), or are coated with a biocompatible coating, for example, a parylene coating.

1-20. (canceled)

21. An allergy test device for applying a liquid medium onto or into skin, comprising:
a film with a flat part for laying on the skin and at least one spaced part spaced apart from the skin during use; and
a container for receiving the liquid medium associated with the at least one spaced part;
wherein the container of the at least one part has at least one closable opening;
wherein the at least one spaced part is deformable in a direction of a flat part of the film and a valve is slidably arranged in the at least one closable opening; and
wherein the at least one closable opening is sealed in a closed position of the valve and the valve is at least partly opened in a position deviating from the closed position when the at least one spaced part is deformed.

22. The allergy test device according to claim 21, wherein the container is integral with the at least one spaced part of the film.

23. The allergy test device according to claim 22, further comprising a filling opening for filling the container with the liquid medium arranged in the at least one spaced part.

24. The allergy test device according to claim 23, wherein the filling opening is configured to be closed with a lid.

25. The allergy test device according to claim 21, wherein the container is formed by a cup connected to the at least one spaced part of the film.

26. The allergy test device according to claim 21, wherein the at least one spaced part of the film or the entire film is elastically deformable.

27. The allergy test device according to claim 26, wherein the film is made of one of thermoplastic synthetic material and silicone.

28. The allergy test device according to claim 21, wherein the at least one spaced part is formed by a protuberance of the film, so that during use a cavity is formed between the film and the skin.

29. The allergy test device according to claim 28, wherein the cavity formed during use is a closed cavity.

30. The allergy test device according to claim 28, wherein a vent hole is arranged in the protuberance.

31. The allergy test device according to claim 21, further comprising at least one pointed or sharp element for local and superficial injury to the skin on at least one of a side of the container and the valve facing the skin during use.

32. The allergy test device according to claim 31, where the at least one pointed or sharp element is made in one piece with at least one of the container and the valve.

33. The allergy test device according to claim 21, wherein:
the valve comprises a substantially cylindrical body with at least one channel;
the cylindrical body in the closed position of the valve sealing the at least one closable opening of the container; and
the cylindrical body in a position deviating from the closed position opening at least one channel to the container.

34. The allergy test device according to claim 33, wherein the at least one channel runs inside the cylindrical body with a connection to the at least one closable opening on a lateral surface of the cylindrical body.

35. The allergy test device according to claim 33, wherein the at least one channel runs on a surface of the cylindrical body.

36. The allergy test device according to claim 33, wherein a sealing lip for sealing the at least one closable opening of the container in the closed position is integrated on the cylindrical body.

37. The allergy test device according to claim 21, further comprising an adhesive film with a peel-off film on a side of the flat part of the film facing the skin during use.

38. The allergy test device according to claim 21, wherein several spaced parts of a film are arranged in at least two rows.

39. The allergy test device according to claim 38, wherein a number of the spaced parts of the film in each row is different.

40. The allergy test device according to claim 21, wherein an identification is arranged on an outside of at least one of the at least one spaced part.

41. The allergy test device according to claim 21, wherein the film is formed at least partly from transparent material.

42. The allergy test device according to claim 21, further comprising a transport lock having substantially cylindrical elements, the transport lock being arranged on an underside of the film for enclosing the container and the valves in the at least one spaced part.

43. The allergy test device according to claim 37, wherein a ruler is arranged on the transport lock for measuring allergic reactions on the skin.

44. The allergy test device according to claim 43, wherein the ruler comprises semi-circular recesses.

45. The allergy test device according to claim 21, wherein sterile packaging is provided.

46. The allergy test device according to claim 28, wherein at least one of the film, the container, the valve and the at least one pointed or sharp element is one of at least partly formed from a biocompatible material and at least partly coated with a biocompatible coating.

47. The allergy test device according to claim 46, wherein at least one of the film, the container, the valve, and the at least one pointed or sharp element is one of:
at least partly formed from polyether ether ketone (PEEK), and
at least partly coated with a parylene coating.
